(19) United States
(12) Patent Application Publication
(10) Pub. No.: US 2024/0424228 A1
(43) Pub. Date: Dec. 26, 2024

(54) Drug Delivery Device

(71) Applicant: SANOFI, Paris (FR)

(72) Inventor: Michael Helmer, Frankfurt am Main (DE)

(21) Appl. No.: 18/700,313

(22) PCT Filed: Nov. 1, 2022
(86) PCT No.: PCT/EP22/80412

§ 371 Date: Apr. 11, 2024

(30) Foreign Application Priority Data
Nov. 4, 2021 (EP) 21315234.1

(51) Int. Cl. A61M 5/50; A61M 5/24

(52) U.S. Cl. CPC A61M 5/50; A61M 2005/2485; A61M 2205/273

(57) ABSTRACT

The disclosure relates to a disposable drug delivery device for selecting and dispensing a number of user variable doses of a medicament. The device comprises an outer housing, an inner housing, a cartridge holder for retaining a cartridge containing the medicament, a piston rod displaceable relative to the cartridge holder, a driver coupled to the piston rod, a display member for indicating a set dose and being coupled to the housing and to the driver, and a button coupled to the display member and to the driver. Engagement of the inner housing in the outer housing may be improved by the inner housing is axially retained in the outer housing by an interface comprising at least one radially deflectable arm with a lateral protrusion, at least one recess adapted for engagement with the protrusion and at least one locking member adapted for hindering disengagement of the at least one protrusion from the at least one recess.

### CROSS REFERENCE TO RELATED APPLICATIONS

The present application is the national stage entry of International Patent Application No. PCT/EP2022/080412, filed on Nov. 1, 2022, and claims priority to Application No. EP 21315234.1, filed on Nov. 4, 2021, the disclosures of which are incorporated herein by reference.

### TECHNICAL FIELD

The present disclosure is generally directed to drug delivery devices. More particularly, the present disclosure is directed to disposable drug delivery devices.

### BACKGROUND

Pen type drug delivery devices have application where regular injection by persons without formal medical training occurs. This may be increasingly common among patients having diabetes where self-treatment enables such patients to conduct effective management of their disease. In practice, such a drug delivery device allows a user to set and dispense a number of doses of a medicament.

There are basically two types of drug delivery devices: resettable devices (i.e., reusable) and non-resettable (i.e., disposable). For example, disposable pen delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism. An example of such a disposable drug delivery device is known from EP 2 890 434 B1 which is incorporated herein by reference regarding the general design and functions of the individual component parts and their interaction during use of the drug delivery device, especially for dose setting and dose dispensing. U.S. Pat. No. 8,652,100 B1 discloses a re-usable drug delivery device comprising a flexible arm and a latch spring forming a releasable lock permitting detachment of an inner housing from the device.

These types of pen delivery devices (so named because they often resemble an enlarged fountain pen) are generally comprised of three primary elements: a cartridge section that includes a cartridge often contained within a housing or holder; a needle assembly connected to one end of the cartridge section; and a dosing section connected to the other end of the cartridge section. A cartridge (often referred to as an ampoule) typically includes a reservoir that is filled with a medication (e.g., insulin), a movable rubber type bung or stopper located at one end of the cartridge reservoir, and a top having a pierceable rubber seal located at the other, often necked-down, end. A crimped annular metal band is typically used to hold the rubber seal in place. While the cartridge housing may be typically made of plastic, cartridge reservoirs have historically been made of glass.

The needle assembly is typically a replaceable double-ended needle assembly. Before an injection, a replaceable double-ended needle assembly is attached to one end of the cartridge assembly, a dose is set, and then the set dose is administered. Such removable needle assemblies may be threaded onto, or pushed (i.e., snapped) onto the pierceable seal end of the cartridge assembly.

The dosing section or dose setting mechanism is typically the portion of the pen device that is used to set a dose. During an injection, a spindle or piston rod contained within the dose setting mechanism presses against the bung or stopper of the cartridge. This force causes the medication contained within the cartridge to be injected through an attached needle assembly. After an injection, as generally recommended by most drug delivery device and/or needle assembly manufacturers and suppliers, the needle assembly is removed and discarded.

Disposable and reusable drug delivery devices have certain perceived disadvantages. One perceived disadvantage is that such devices have a high number of parts and therefore such devices are typically complicated from a manufacturing and from an assembly standpoint. In addition, because such devices use a large number of components parts, such devices tend to be large and bulky, and therefore not easy to carry around or easy to conceal.

To avoid misuse of the drug delivery device, it is desirable to prevent a user from dissembling the device. This is especially important for disposable drug delivery devices which are intended to be discarded after use, e.g., when the cartridge is empty. Attempts to replace or refill an empty cartridge by dissembling the device may cause severe health issue due to inappropriate dosage or impurities and may result in malfunction of the device. Disassembly of such a device typically requires separation of the inner housing from the outer housing to allow access to the cartridge or to reset limiter functions of the device.

In the drug delivery device disclosed in EP 2 890 434 B1, an inner housing is fixed into an outer housing by means of an annular bead on an outer surface of the inner housing engaging an annular groove on an inner surface of the outer housing. In certain scenarios, e.g., if excessive forces are applied to the device in case of a blocked needle or in case of an attempt to dissemble the device, this interface between the inner housing and the outer housing may disengage.

### SUMMARY

The present disclosure relates to an improved drug delivery device, especially regarding the robustness to withstand unintended dissembling.

In particular, the present disclosure relates to a disposable drug delivery device for selecting and dispensing a number of user variable doses of a medicament, comprising an inner housing, an outer housing, a cartridge holder for retaining a cartridge containing the medicament, a piston rod displaceable relative to the cartridge holder, a driver coupled to the piston rod, a display member for indicating a set dose and being coupled to the inner housing and to the driver, and a button coupled to the display member and to the driver. The risk of unintended or undesired disassembly of the device may be reduced by axially retaining the inner housing in the outer housing. The disposable drug delivery device includes an interface comprising at least one radially deflectable arm with a lateral protrusion, at least one recess adapted for engagement with the protrusion and at least one locking member adapted for hindering (i.e. for preventing) disengagement of the at least one protrusion from the at least one recess.

Such an interface may be provided in addition to the annular bead and the annular groove known from the device disclosed in EP 2 890 434 B1 or as an alternative to the bead and groove. The present disclosure is based on the idea that disassembly of the inner housing from the outer housing may be achieved by elastic or limited plastic deformation of the housing components, especially in the area of the bead and the groove in a device as disclosed in EP 2 890 434 B1. The provision of an arm engaging a respective recess for axially locking the inner housing in the outer housing may increase the forces required for disassembling the inner housing from the outer housing compared to an interface comprising a bead and a groove. In addition, a locking member preventing or at least hindering disengagement of the protrusion from the recess reduces the risk of unintended or undesired disassembly. In other words, while the arm with the protrusion engaging the recess provides for a strong mechanical interlocking of the inner housing in the outer housing, the locking member contributes to keeping the protrusion and the recess in the engaged configuration.

According to the present disclosure, a disposable drug delivery device is a drug delivery device in which a cartridge is provided in a cartridge holder such that the cartridge holder and the cartridge may not be removed and replaced from the device without destroying the device itself. In other words, the cartridge holder of a disposable drug delivery device is either a one-piece component part with the, e.g., outer, housing of the drug delivery device or the cartridge holder is permanently attached to the, e.g., outer, housing, thereby preventing detachment of the cartridge holder.

The outer housing of the drug delivery device defines a central axis extending between a distal end and a proximal end. “Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example. The terms “axial”, “radial”, or “circumferential” as used herein may be used with respect to a main longitudinal axis of the device, the cartridge, the housing or the cartridge holder, e.g. the axis which extends through the proximal and distal ends of the cartridge, the cartridge holder or the drug delivery device.

According to a further aspect of the present disclosure the inner housing may comprise the at least one radially deflectable arm and the outer housing may comprise the at least one recess. For example, two deflectable arms and recesses may be provided. More than two arms and recesses could be designed if a higher disassembly force is desired. With such a configuration, the lateral protrusion may be directed radially outwards. For example, the inner housing may comprise a distally facing substantially cylindrical engagement portion wherein the at least one radially deflectable arm may be integrally formed in the engagement portion. Further, the at least one recess may be a radial aperture in the outer housing. To prevent dirt from entering the drug delivery device through this radial aperture in the outer housing, the aperture may be covered by a label, shield and/or sleeve provided on an outer surface of the outer housing. In addition, this interface may function as a tamper-evident closure. In other words, the device components may be provided such that they would be damaged by cartridge replacement. For example, to remove an inner body component from an outer body component a final assembled label may have to be removed or damaged, e.g. in the areas of the protrusions.

The lateral protrusion of the arm may be formed facilitating assembly of the device but hindering disassembly. For example, the protrusion may form a snap hook engaging a corresponding counter portion of the recess. The protrusion may be ramped or inclined on a distally facing side such that the arm with the protrusion is deflected radially inwards as the inner housing is inserted into the outer housing. On the other hand, the proximally facing side of the protrusion may be formed or inclined such that an axial force aiming at retracting the inner housing from the outer housing does not cause significant forces biasing the arm radially inwards or may even cause forces biasing the arm further outwards, e.g., increasing the locked engagement.

The at least one locking member may be or may comprise a pin, e.g. a pin provided on a radially inwardly facing side of the at least one arm. This pin may be displaced towards component parts located radially inwards of the arm, thus preventing radially inwards directed deflection of the arm. For example, at least a proximal end of the cartridge is retained in the inner housing, e.g. in distally facing substantially cylindrical engagement portion of the inner housing. Hence, abutment of the pin and the cartridge may prevent radially inwards directed deflection of the arm.

According to a further aspect of the present disclosure the outer housing may comprise the at least one radially deflectable arm and the inner housing may comprise the at least one recess. Two or more deflectable arms and recesses may be provided, e.g., if a higher disassembly force is desired. With such a configuration, the lateral protrusion my be directed radially inwards. For example, the outer housing may comprise a substantially cylindrical engagement portion and the at least one radially deflectable arm may be integrally formed in the engagement portion. Further, the at least one recess may be a radial aperture in the inner housing.

Although the provision of the arm in the outer housing does not necessarily cause large lateral openings in the outer housing, it may be favourable to cover the at least one radially deflectable arm of the outer housing by a label, shield and/or sleeve provided on an outer surface of the outer housing to prevent dirt from entering the drug delivery device through this radial aperture in the outer housing. With this configuration, the at least one locking member may be formed by said label, shield and/or sleeve as it prevents or at least hinders outwards deflection of the arm which would be required for disengagement of the protrusion from the recess and, thus, detachment of the inner housing from the outer housing.

Again, the lateral protrusion of the arm may be formed facilitating assembly of the device but hindering disassembly. For example, the protrusion may form a snap hook engaging a corresponding counter portion of the recess. The protrusion may be ramped or inclined on a distally facing side such that the arm with the protrusion is deflected radially outwards as the inner housing is inserted into the outer housing. On the other hand, the proximally facing side of the protrusion may be formed or inclined such that an axial force aiming at retracting the inner housing from the outer housing does not cause significant forces biasing the arm radially outwards or may even cause forces biasing the arm further inwards, i.e. increasing the locked engagement.

The outer housing of the drug delivery device may comprise an inner shoulder between a proximal portion having a larger diameter and a distal portion having a smaller diameter. The distal end face of the inner housing may rest against the shoulder of the outer housing to form a distal end stop for the inner housing. In other words, distal movement of the inner housing relative to the outer housing is prevented by the shoulder, whereas proximal movement of the inner housing relative to the outer housing is prevented by the engagement of the protrusion in the recess of the interface.

In this respect, it may be advantageous to provide the cartridge holder as a one-piece component with the outer housing. Combining the cartridge holder and the outer housing into one single component reduces the number of component parts and thus assembling complexity. Further, the risk of a misuse, where a user attempts to exchange an empty cartridge in a disposable drug delivery device, is reduced.

In addition to the axial alignment and fixing of the inner housing in the outer housing, the inner housing may be rotationally constrained to the outer housing. For example, the interface may further comprise at least one axially extending rib and at least one corresponding axially extending groove for rotationally securing the inner housing in the outer housing.

The drug delivery device may further comprise a container receptacle which is releasably attached to the dose setting and drive mechanism. As an alternative, the container receptacle may be permanently attached to the dose setting and drive mechanism. The container receptacle is adapted to receive a container, e.g., a cartridge, containing a medicament.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively, or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12(anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Chorion-gonadotropin, Menotropin), Somatropine (Somatropin), Desmopressin, Terlipressin, Gonadorelin, Triptorelin, Leuprorelin, Buserelin, Nafarelin, and Goserelin. Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate. The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full-length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF FIGURES

Non-limiting, exemplary embodiments of the disclosure will now be described with reference to the accompanying drawings, in which:

FIG. 1 shows a perspective view of a prior art drug delivery device;

FIG. 2 shows an exploded view of the prior art drug delivery device of FIG. 1;

FIG. 3 schematically illustrates a sectional view of a detail of a drug delivery device according to a first embodiment of the present disclosure; and

FIG. 4 schematically illustrates a sectional view of a detail of a drug delivery device according to a second embodiment of the present disclosure.

### DETAILED DESCRIPTION

In the figures, identical elements, identically acting elements or elements of the same kind may be provided with the same reference numerals.

In the following, some embodiments will be described with reference to an insulin injection device. The present disclosure is however not limited to such application and may equally well be deployed with injection devices that are configured to eject other medicaments or drug delivery devices in general, including pen-type devices and/or injection devices.

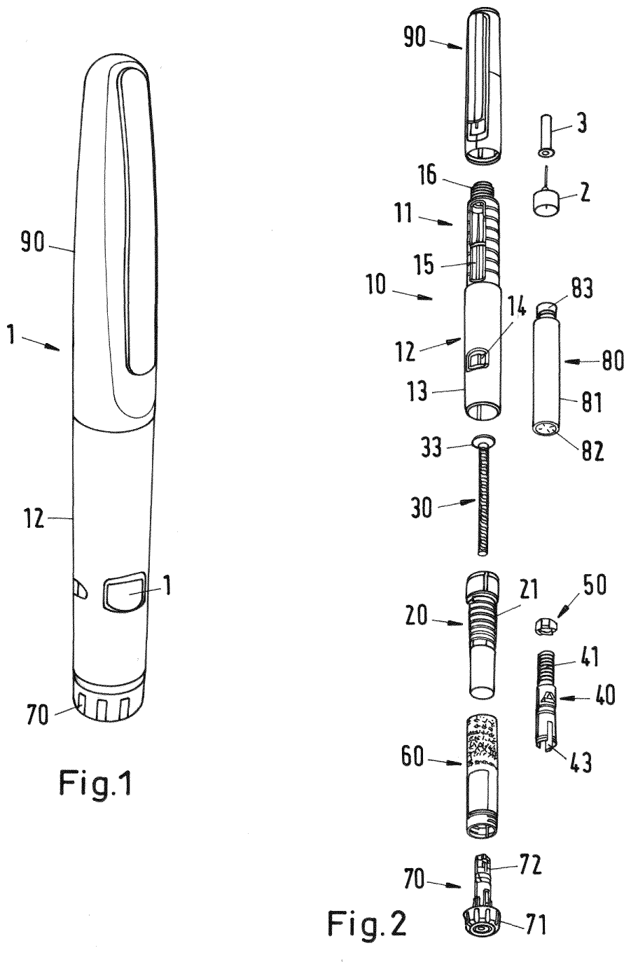

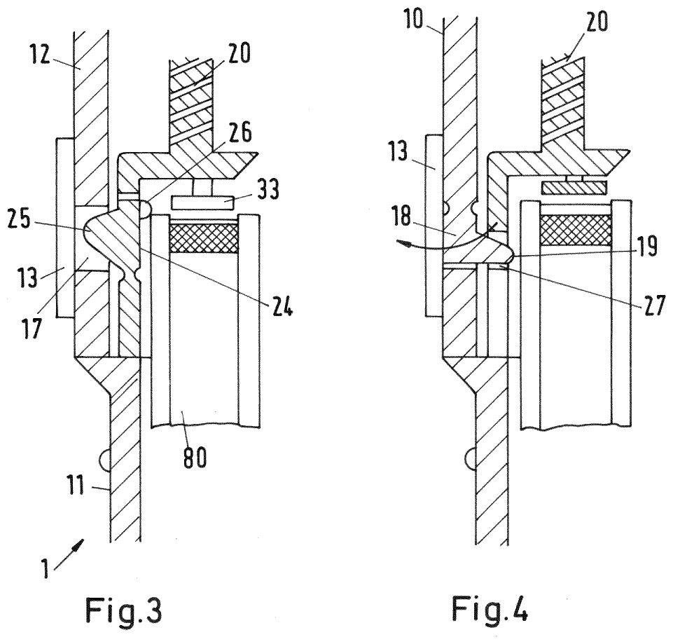

FIGS. 1 and 2 show a perspective view and an exploded view of the medicament delivery device or drug delivery device disclosed in EP 2 890 434 B1. FIGS. 3 and 4 depict improvements according to the present disclosure which are especially applicable to the device of FIGS. 1 and 2.

FIG. 1 shows the drug delivery device 1 in the form of an injection pen. The device has a distal end (upper end in FIG. 1) and a proximal end (lower end in FIG. 1). The component parts of the drug delivery device 1 are shown in FIG. 2 in more detail. The drug delivery device 1 comprises an outer housing 10, an inner housing 20, a piston rod 30, a driver 40, a nut 50, a display member 60, a button 70, a cartridge 80 and a cap 90, e.g., in total nine separate component parts. As shown in FIG. 2, a needle arrangement comprising a needle hub 2 and a needle cover 3 may be provided as additional components, which can be exchanged as explained above.

The outer housing 10 is a generally tubular element having a distal part, which forms a cartridge holder 11 for receiving cartridge 80, and a proximal part, which forms an outer body 12 receiving components for dose setting and dose dispensing. The outer housing 10 may be transparent, with the outer body 12 being provided with an, e.g., opaque, label (e.g., layer) 13 which may be a label, shield and/or sleeve. In FIG. 2, the label (e.g., layer) 13 covers most of the outer body 12 with the exception of a transparent window 14. Apertures 15 may be provided in the cartridge holder 11. Further, at its distal end the cartridge holder 11 has a thread 16 or the like for attaching the needle hub 2.

The inner housing 20 is a generally tubular element having different diameter regions. As can be seen in FIGS. 2 to 4, the inner housing 20 is received in the outer body 12 and permanently fixed therein to prevent any relative movement of the inner housing 20 with respect to the outer housing 10. An external thread 21 is provided on the outer surface of the inner housing 20. Further, splines may be provided on the inner surface of the inner housing 20 (not shown). An axially extending rib 22 of the inner housing 20 may engage a corresponding axial groove (not shown) in the outer housing 10. In addition, an annular bead 23 is provided near the distal end of the inner housing 20 which may engage a corresponding annular groove (not shown) of the outer housing 10.

The piston rod 30 is an elongate element having two external threads with opposite hand which overlap each other. One of these threads engages an inner thread of the inner housing 20. A disk-like bearing 33 is provided at the distal end of the piston rod 30. As shown in FIG. 2, the bearing 33 may be attached to the piston rod 30 as a one-piece component via a predetermined breaking point. This allows that the bearing 33 is separated from the piston rod 30 such that the bearing 33 remains seated on the distal end of the piston rod 30 to allow relative rotation between the bearing 33 and the piston rod 30.

The driver 40 is a generally tubular element having different diameter regions. A distal region of the driver 40 has an external thread 41. An inner surface of the driver 40 has an inner thread (not shown) engaging one of the external threads of the piston rod 30. The driver 40 surrounds the piston rod 30 and is at least partly located within inner housing 20. The driver has at least one proximal opening 43. Further, a resilient finger may be provided on the driver 40 by a U-shaped cut in the skirt of the driver 40. The finger is allowed to flex in the axial direction and engages button 70. In addition, a flexibly hinged protrusion may be provided on the driver 40 by a similar cut out in the skirt of the driver 40. The protrusion is allowed to flex radially inwardly and engages inner splines of the inner housing 20.

The nut 50 is provided between the inner housing 20 and the driver 40. External ribs of the nut 50 engage splines of the inner housing 20. An internal thread of the nut 50 engages the external thread 41 of the driver 40.

The display member 60 is a generally tubular element with an internal thread engaging the external thread 21 of the inner housing 20. Thus, the display member 60 is interposed between the inner housing 20 and the outer body 12. A series of numbers is provided, e.g., printed, on the outer surface of the display member 60. The numbers are arranged on a helical line such that only one number or only a few numbers are visible in through window 14 of the outer body 12. The display member 60 is attached to the driver 40 preventing relative axial movement but allowing relative rotation.

The button 70 has a proximal end with an, e.g., serrated, flange or outer skirt 71 allowing a user to easily grip and dial button 70. A sleeve-like part 72 of the button 70 with a reduced diameter extends in the distal direction and is inserted into the driver 40 such that a limited relative axial movement is allowed but relative rotation is prevented. This may be achieved by a rib on the sleeve-like part 72 which is guided in the proximal opening 43 of the driver 40.

A clutch is provided between the display member 60 and the button 70 by corresponding teeth (not shown). If the teeth of the button 70 engage the teeth of the display member 60, these components are rotationally locked. The resilient finger of the driver 40 may bias the button 70 in the proximal direction of the device 1, i.e., in a direction engaging the clutch teeth. The clutch can be released allowing relative rotation by shifting the button 70 axially with respect to the display member 60 against the bias of this finger.

The cartridge 80 includes a pre-filled, necked-down cartridge reservoir 81, which may be typically made of glass. A rubber type bung 82 or stopper is located at the proximal end of the cartridge reservoir 81, and a pierceable rubber seal (not shown) is located at the other, distal, end. A crimped annular metal band 83 is used to hold the rubber seal in place. The cartridge 80 is provided within the cartridge holder 11 with bearing 33 of piston rod 30 abutting bung 82.

FIG. 1 shows the cap 90 attached to the distal end of the device 1, thus covering the cartridge holder 11. The cap 90 may be releasable snapped onto the outer housing 10 and can be taken off for use of the device 1.

Regarding the function of the disposable drug delivery device 1 and its components reference is made to EP 2 890 434 B1.

FIG. 3 shows a sectional view of a portion of the drug delivery device 1 according to a first embodiment of the present disclosure. Unless specified in the following, the component parts and their functions are identical as described above for FIGS. 1 and 2.

In FIG. 3, the inner housing 20 is provided with an arm 24 which is integrally formed in a distal portion of the inner housing 20, e.g., by a U-shaped cut in the skirt of the inner housing 20. As depicted, a free end of arm 24 may face in the proximal direction while the distal end of the arm is connected to the inner housing 20. This arm 24, more specifically its free end, may be bent or flexed radially inwards form the unstressed condition as depicted in FIG. 3. A lateral protrusion 25 extends radially outwards from the free end of the arm 24. In addition, an optional pin 26 is provided on a radially inner side of the arm 24. The pin could support cartridge centering into the cartridge holder part and slightly engage the arms into the recess (during pivoting). On the other hand, a high pressure onto the glass edge could cause glass breakage.

As depicted, the inner housing 20 may have a distal engagement portion with an increased diameter and a proximal portion with the external thread 21.

The outer housing 10 is provided with an inner shoulder in the area where the cartridge holder 11 portion of the outer housing 10 merges with the outer body 12 portion. The distal end of inner housing 20 rests against this shoulder. An aperture in the outer housing 10 forms a recess 17 for receiving protrusion 25 as depicted in FIG. 3. Engagement of protrusion 25 in recess 17 locks the inner housing 20 against proximal movement with respect to the outer housing 10. The label 13 covers the aperture of recess 18 from the outside.

As can be seen in FIG. 3, in the depicted assembled state of the drug delivery device 1 the position of the proximal end of cartridge 80 is such that the pin 26 would interfere with the cartridge 80 if the arm 24 would flex radially inwards. Thus, the pin 26 in conjunction with the cartridge 80 prevents the arm 24 from bending inwards and, thus, prevents disengagement of the protrusion 25 from the recess 17. Thus, pin 26 functions as a locking member for the interface of the arm 24 and the recess 17.

An alternative second embodiment is depicted in FIG. 4 which shows a sectional view of a portion of the drug delivery device 1. Again, unless specified in the following, the component parts and their functions are identical as described above for FIGS. 1 and 2.

The drug delivery device 1 depicted in FIG. 4, has an arm 18 with an inwardly facing protrusion 19 integrally formed in the outer housing 10, namely in outer body 12, e.g., by a U-shaped cut in the skirt of the outer housing 10. In this embodiment, the inner housing 20 is provided with a recess 27 formed by a lateral aperture. In a similar manner as described above with respect to FIG. 3, the outer housing 10 is provided with an inner shoulder in the area where the cartridge holder 11 portion of the outer housing 10 merges with the outer body 12 portion. The distal end of inner housing 20 rests against this shoulder. Engagement of protrusion 19 in recess 27 locks the inner housing 20 against proximal movement with respect to the outer housing 10. The label 13 covers the arm 18 from the outside, thereby preventing that the arm 18 is flexed outwards as would be required for disengagement of the protrusion 19 from the recess 27. Thus, label 13 functions as a locking member for the interface of the arm 18 and the recess 27.

With the interface locking the inner housing 20 axially to the outer housing 10 as described with reference to the embodiments depicted in FIGS. 3 and 4, disengagement of the inner housing 20 from the outer housing 10 under undesired use scenarios, e.g., applying a dispensing force with a blocked needle, is prevented. This locked snap fit like interface between the inner housing 20 and the outer housing 10 is further suitable to withstand misuse scenarios aiming at disassembling the device 1 by applying an axial force on the inner housing 20 in an attempt to pull or push it out of the outer housing 10.

Reference numerals:

| | |
|---|---|
| 1 | drug delivery device |
| 2 | needle hub |
| 3 | needle cover |
| 10 | outer housing |
| 11 | cartridge holder |
| 12 | outer body |
| 13 | layer (label) |
| 14 | window |
| 15 | aperture |
| 16 | thread |
| 17 | recess |
| 18 | arm |
| 19 | protrusion |
| 20 | inner housing |
| 21 | thread |
| 22 | rib |
| 23 | bead |
| 24 | arm |
| 25 | protrusion |
| 26 | pin |
| 27 | recess |
| 30 | piston rod |
| 33 | bearing |
| 40 | driver |
| 41 | thread |
| 43 | opening |
| 50 | nut |
| 60 | display member |
| 70 | button |
| 71 | skirt |
| 72 | sleeve-like part |
| 80 | cartridge |
| 81 | reservoir |
| 82 | bung |
| 83 | band |
| 90 | cap |

1-15. (canceled)

16. A disposable drug delivery device for selecting and dispensing a number of doses of a medicament, the disposable drug delivery device comprising:
an outer housing defining a central axis extending between a distal end and a proximal end;
an inner housing;
a cartridge holder for retaining a cartridge containing the medicament;
a piston rod displaceable relative to the cartridge holder,
a driver coupled to the piston rod;
a display member for indicating a set dose and being coupled to the inner housing and to the driver; and
a button coupled to the display member and to the driver, wherein the inner housing is axially retained in the outer housing by an interface comprising at least one radially deflectable arm with a lateral protrusion, at least one recess adapted for engagement with the lateral protrusion and at least one locking member adapted for hindering disengagement of the lateral protrusion from the at least one recess.

17. The disposable drug delivery device of claim 16, wherein the inner housing comprises the at least one radially deflectable arm, wherein the lateral protrusion is directed radially outwards, and wherein the outer housing comprises the at least one recess.

18. The disposable drug delivery device of claim 17, wherein the inner housing comprises a distally facing substantially cylindrical engagement portion and wherein the at least one radially deflectable arm is integrally formed in the engagement portion.

19. The disposable drug delivery device of claim 18, wherein the at least one recess is a radial aperture in the outer housing.

20. The disposable drug delivery device of claim 19, wherein the radial aperture in the outer housing is covered by a label, shield and/or sleeve provided on an outer surface of the outer housing.

21. The disposable drug delivery device of claim 19, wherein the at least one locking member is a pin provided on a radially inwardly facing side of the at least one radially deflectable arm.

22. The disposable drug delivery device of any of claim 19, wherein at least a proximal end of the cartridge is retained in the inner housing by a distally facing substantially cylindrical engagement portion of the inner housing (20).

23. The disposable drug delivery device of claim 16, wherein the outer housing comprises the at least one radially deflectable arm, wherein the lateral protrusion is directed radially inwards, and wherein the inner housing comprises the at least one recess.

24. The disposable drug delivery device of claim 23, wherein the outer housing comprises a substantially cylindrical engagement portion and wherein the at least one radially deflectable arm is integrally formed in the engagement portion.

25. The disposable drug delivery device of claim 23, wherein the at least one recess is a radial aperture in the inner housing.

26. The disposable drug delivery device of claim 23, wherein the at least one radially deflectable arm of the outer housing is covered by a label, a shield and/or a sleeve provided on an outer surface of the outer housing.

27. The disposable drug delivery device of claim 26, wherein the at least one locking member is formed by the label, the shield and/or the sleeve.

28. The disposable drug delivery device of claim 16, wherein the outer housing comprises an inner shoulder between a proximal portion and distal portion which distal portion has a smaller inner diameter than the proximal portion.

29. The disposable drug delivery device of claim 28 wherein a distal end face of the inner housing rests against the inner shoulder of the outer housing.

30. The disposable drug delivery device of any of claim 16, wherein the cartridge holder is a one-piece component with the outer housing.

31. The disposable drug delivery device of claim 16, wherein the interface further comprises at least one axially extending rib and at least one corresponding axially extending groove for rotationally securing the inner housing in the outer housing.

32. The disposable drug delivery device of claim 16, further comprising a cartridge containing a medicament.

33. The disposable drug delivery device of claim 16, further comprising a clutch between the display member and the button, the clutch comprising teeth rotationally locking the display member and the button, the clutch being releasable by axially shifting the button with respect to the display member.

34. The disposable drug delivery device of claim 16, wherein the display member comprises a series of numbers on an outer surface of the display member.

35. The disposable drug delivery device of claim 34, wherein the series of numbers are arranged on a helical line on the outer surface of the display member.
